(19) United States
(12) Patent Application Publication
(10) Pub. No.: US 2015/0272855 A1
(43) Pub. Date: Oct. 1, 2015

(54) COSMETIC SHEET FORMED FROM NANOFIBER WITH CONTROLLED DISSOLUTION VELOCITY AND METHOD OF MANUFACTURING THE SAME

(71) Applicant: AMOGREENTECH CO., LTD., Gimpo-si Gyeonggi-do (KR)

(72) Inventor: Chan Kim, Gwangju (KR)

(73) Assignee: AMOGREENTECH CO., LTD., Gimpo-si Gyeonggi-do (KR)

(21) Appl. No.: 14/437,234

(22) PCT Filed: Oct. 23, 2013
(86) PCT No.: PCT/KR2013/009459

§ 371 Date: Apr. 21, 2015

(30) Foreign Application Priority Data
Oct. 25, 2012 (KR) 10-2012-0118984

(51) Int. Cl. A61K 8/81; A61K 8/02; A61Q 19/00; A61K 8/73; A61K 8/92; A45D 37/00; A61K 8/65

(52) U.S. Cl. CPC A61K 8/8182; A45D 37/00; A61K 8/0204; A61K 8/8129; A61K 8/65; A61K 8/8176; A61K 8/735; A61K 8/922; A61Q 19/00; A61K 8/0208

(57) ABSTRACT

Provided is a cosmetic sheet formed from nanofiber that is obtained by preparing a spinning solution that is formed by dissolving a water-soluble polymer material together with a functional material in a solvent of water or alcohol, and electrospinning the spinning solution wherein the nanofiber has a controlled dissolution velocity and has fine pores. The water-soluble nanofiber layer can control a dissolution velocity by moisture or mist through crosslinking. The attached cosmetic sheet is a soluble melt-type such that the cosmetic sheet is naturally melted and absorbed into the skin, thereby being used in everyday life without having to be removed separately, and having excellent thin and adhesive properties.

### TECHNICAL FIELD

The present invention relates to a cosmetic sheet and a method of manufacturing the same, and more particularly, to a cosmetic sheet formed from nanofiber obtained by electrospinning a water-soluble polymer together with a functional material to thus be melted by the moisture and absorbed in the skin when adhering closely to the skin so that there is no need to be removed separately from the skin, and a method of manufacturing the same.

### BACKGROUND ART

Typical cosmetic sheets contain essences including nutrient materials such as natural extracts, proteins, vitamins on nonwoven fabrics, so as to have functions such as whitening, anti-wrinkle, moisturizing, skin irritation relief, skin elasticity enhancement, and antibacterial activity.

However, since typical cosmetic sheets are prepared on the basis of non-woven fabrics, sufficient adhesion is not achieved on the interface to contact the skin, and thus efficacious ingredients are not delivered sufficiently to the depths of the skin. As a result, it has been attempted to improve close adhesion or attachment to the skin by the addition of an excess of essences.

Therefore, the typical cosmetic sheets may cause inconvenience of use and unpleasant feelings by the falling down of the typical cosmetic sheets due to the dead weight of the typical cosmetic sheets, or the flowing down of the excessive essences, and cause disadvantages of wasting the essences. Further, in the case of wearing typical cosmetic sheets, it may be difficult to do daily activities and cause constraints of activities such that wearers should be laid down during wearing of the cosmetic sheets.

To solve this drawback, hydrogel cosmetic sheets have recently been used a lot. The hydrogel cosmetic sheets have excellent fit and allow for doing daily activities, but are thick not only to cause limitation to the close adhesion thereof, but also to cause inconveniences of separately removing the hydrogel cosmetic sheets off after wearing time has passed.

Recently, researches on an electrospinning method as a method of forming nanofiber having a diameter of less than 1 μm are being actively investigated. As soon as nanofiber is produced by the electrospinning method, they are formed in a laminated structure having a three-dimensional fine pore structure, to thereby be applicable to various fields including industrial or medical fields such as various filter materials, ultra-lightweight functional clothes utilizing waterproof breathable functions, materials using pore characteristics and large surface areas for biomedical use, and inorganic materials and carbon materials through post-processes.

FIG. 1 schematically illustrates a simulation of a contact surface between fiber and the skin when microfiber with a diameter of 20 μm and nanofiber with a diameter of 0.2 μm (200 nm) are in contact with the skin, respectively. That is, while about 500 strands of fiber per unit area are in contact with the skin in the case of the non-woven fabric with a diameter of 20 μm, about 50,000 strands of fiber per unit area are in contact with the skin in the case of the nanofiber, and thus nanofiber shows a contact area of at least about 10,000-fold in comparison with the non-woven fabric. Thus, the finer the diameter of fiber may be, the greater the contact area with the skin may be maximized. Therefore, when skin cosmetic sheets are prepared by using nanofiber, the contact area with the skin becomes much larger than the contact area of the skin in the skin cosmetic sheets prepared based on an existing non-woven fabric or cloth, to thereby improve the adhesion to the skin.

Korean Patent Application Publication No. 10-2011-080066 disclosed skin cosmetic sheets in which a double layer of a nanofiber layer is formed on a nonwoven fabric, and the nanofiber layer of the double layer is surface-treated with plasma. However, since this technique includes a process of compounding the nanofiber layer on the non-woven fabric and a secondary process such as a plasma treatment, processing cost may increase, and since the non-woven fabric is involved, a problem of causing discomfort in wearing the skin cosmetic sheets and doing activities still remains.

Further, a process such as lamination using chemical adhesives, thermal bonding or ultrasonic bonding is required to compound the nonwoven fabric and the nanofiber layer. When the adhesives and so on are not used, a peel is also likely to occur between the non-woven fabric and the nanofiber layer by functional essences, water or the like.

Furthermore, when nanofiber of a double layer structure is spun in a core/shell morphology, a core portion is made of polyurethane and a cell portion being in contact with the skin is made of a biodegradable polymer in order to minimize skin troubles. However, since a solvent of toxicity is used, if the residual solvent is not removed completely, a problem of secondary pollution may be caused due to the residual solvent.

In particular, most of the biodegradable polymers used in the conventional techniques are required to undergo a hydrophilic treatment through a plasma process because of their hydrophobic properties. As a result, processing cost may increase, and a problem of causing a deterioration of the functional material loaded during spinning may also remain.

### DISCLOSURE

### Technical Problem

To solve the above problems or defects of the conventional cosmetic sheets as described above, it is an object of the present invention to provide a cosmetic sheet formed from nanofiber with a controlled dissolution velocity so that an efficacious ingredient can be transmitted properly to the skin by making a layer of nanofiber obtained by electrospinning a water-soluble polymer together with a functional material closely adhere to the skin after washing or mist-spraying the skin, and a method of manufacturing the same.

Further, it is another object of the present invention to provide a cosmetic sheet formed from nanofiber in which a nanofiber layer is dissolved by the water to thus maximize close adhesion and adhesiveness, the cosmetic sheet adhered closely to the skin is automatically melted and absorbed in the skin, to thereby enable daily activities without separately removing the cosmetic sheet from the skin and to thus enhance ease of use, and a method of manufacturing the same.

In another aspect, the present invention has another object to provide a melt-type cosmetic sheet formed from nanofiber in which a problem of a secondary pollution source due to a residual solvent is removed since water or alcohol only is used as the solvent on the basis of a water-soluble polymer, and a method of manufacturing the same.

In another aspect, the present invention has another object to provide a melt-type cosmetic sheet formed from nanofiber in which efficacious ingredients are delivered sufficiently to the skin by controlling a degree of crosslinking of a water-soluble polymer to thereby adjust a velocity of dissolution of the cosmetic sheet by water or mist during adhesion of the cosmetic sheet, and a method of manufacturing the same.

The objects of the present invention are not limited to the above-described objects, and other objects and advantages of the present invention can be appreciated by the following description and will be understood more clearly by embodiments of the present invention.

### Technical Solution

To accomplish the above and other objects of the present invention, according to an aspect of the present invention, there is provided a cosmetic sheet formed from nanofiber having a diameter less than 1 μm with a controlled dissolution velocity and having fine pores, in which the nanofiber is obtained by electrospinning a spinning solution that is formed by dissolving a water-soluble polymer material together with a functional material in a solvent of water or alcohol.

The water-soluble polymer material that is used for preparing the nanofiber in the present invention may contain one or a mixture of two or more selected from among a group of polymer materials consisting of PVA (polyvinyl alcohol), PVP (polyvinyl pyrrolidone), PEO (polyethylene oxide), CMC (carboxyl methyl cellulose), starch, PAA (polyacrylic acid) and hyaluronic acid.

The content (basis weight) of the nanofiber in the present invention is preferably set in a range of 10-50 gsm (gram per square meter). In the case of less than 10 gsm, a handling problem may occur with an excessive thin film. In the case of exceeding 50 gsm, there is no problem in use, but processing costs may rise due to high costs of materials. Therefore, the amount of the water-soluble polymer material which is dissolved in a solvent is determined in view of the basis weight of the resulting nanofiber.

The functional material in the present invention may employ one or a mixture of two or more selected from among a group consisting of a water-soluble collagen, vegetable platinum, tocopherol, xylitol and various plant extracts. The content of addition of the functional material is suitable in a range of 0.5-50 wt % when compared to the water-soluble polymer. In the case of the functional material of less than 0.5 wt %, the content of the functional material is too small to exhibit sufficient effects, whereas in the case of the functional material of exceeding 50 wt %, spinning of the fiber is not carried out smoothly, and thus the functional material can be wasted excessively.

In addition, the solvent used in the present invention may employ, for example, water or alcohol. It is preferable to use a solvent innocuous to the human body even with any residual solvent. Further, the solvent may be used alone or in combination with water and/or alcohol, depending on type of the polymer used.

Further, according to another aspect of the present invention, there is provided a method of manufacturing a cosmetic sheet formed from nanofiber with a controlled dissolution velocity, the method comprising the steps of:

preparing a spinning solution by dissolving a water-soluble polymer material and a functional material in a solvent formed of one of or a mixture of water and alcohol to then be mixed with a crosslinking agent;

obtaining a nanofiber web with a diameter of less than 1 μm by electrospinning the spinning solution;

undergoing a crosslinking treatment after pressing the nanofiber web; and

cutting the crosslinked nanofiber web so as to be fitted on shape of the cosmetic sheet.

The crosslinking of the nanofiber in the present invention can be processed by means of physical or chemical methods. The physical methods may include a heat treatment or a crystallization method, and the chemical methods may include a treatment by the addition of a crosslinking agent (or co-crosslinking agent).

In the present invention, the crosslinking agent (or co-crosslinking agent) may include at least one selected from TSA (para-toluene sulfonic acid), TMPTMA (tri-methylopropane trimethacrylate), DVB (Divinylbenzene), N-(1-Hydroxy-2, 2-dimethoxyethyl) acrylamide, N,N′-Methylenebisacrylamide, ethylene glycol diacrylate, Di(ethylene glycol) diacrylate, boric acid, and glutaraldehyde. The content of the crosslinking agent is suitable in a range of 0.1 to 2 wt % when compared to the water-soluble polymer. When the content of the crosslinking agent is less than 0.1 wt %, there may be fears that sufficient crosslinking may not occur, and crosslinking time may be long. In addition, when the content of the crosslinking agent exceeds 2 wt %, the content of the crosslinking agent is too many to be likely to have the residual crosslinking agent that does not take part in the crosslinking reaction.

In the present invention, crosslinking may preferably employ a method such as hot air, heat-treatment, or UV irradiation that is conducted within a range that deterioration or functional failure of the functional material does not occur over time. Further, it is preferable to achieve partial crosslinking in accordance with a purpose of an intended use.

Meanwhile, the spinning in the present invention may employ any one process selected from among electrospinning, electrospray, electrobrown spinning, centrifugal electrospinning, and flash-electrospinning.

### Advantageous Effects

As described above, the melt-type cosmetic sheet formed from nanofiber according to the present invention can allow the functional material to be mounted on polymer nanofiber having a very large specific surface area, to thus greatly enlarge a contact area with the skin, smoothen effective delivery of the functional material, and improve close adhesion to the skin by spraying water or mist onto the skin.

In addition, the melt-type cosmetic sheet according to the present invention is attached to the skin in the form of a thin film type nanofiber web and has a dissolution feature, to thus enable a wearer who uses the melt-type cosmetic sheet closely adhered to the skin to do daily activities, and to thus have no need to use an excess of essences to thereby obtain an effect of providing an ultra-lightweight cosmetic sheet.

### BRIEF DESCRIPTION OF THE DRAWINGS

FIG. 1 schematically illustrates a simulation of a contact surface between fiber and the skin when microfiber with a diameter of 20 μm and nanofiber with a diameter of 0.2 μm (200 nm) are in contact with the skin, respectively.

FIG. 2 shows a manufacturing procedure of a cosmetic sheet including nanofiber according to the present invention.

FIG. 3 is a scanning electron micrograph (SEM) photo of a PVA nanofiber web produced according to the present invention.

FIG. 4 is a scanning electron micrograph (SEM) photo of a completely crosslinking treated PVA nanofiber web according to an embodiment of the present invention.

FIG. 5A is a scanning electron micrograph (SEM) photo of a PVP nanofiber web produced according to an embodiment of the present invention, and FIG. 5B is a graph showing the distribution of the diameter of the nanofiber of FIG. 5A.

FIG. 6 is a scanning electron micrograph (SEM) photo of a PVP nanofiber web in which a functional material is mounted in accordance with the present invention.

FIGS. 7A and 7B are scanning electron micrograph (SEM) photos respectively having 100-times magnification and 5,000-times magnification of a PVA/PVP composite nanofiber web produced according to the present invention.

FIGS. 8A, 8B and 8C are photos showing the degrees a PVA/PVP nanofiber web produced according to an embodiment of the present invention is dissolved in water depending on respective contact times of 3 seconds, 10 seconds, and 30 seconds at the time of heat-calendering the PVA/PVP nanofiber web.

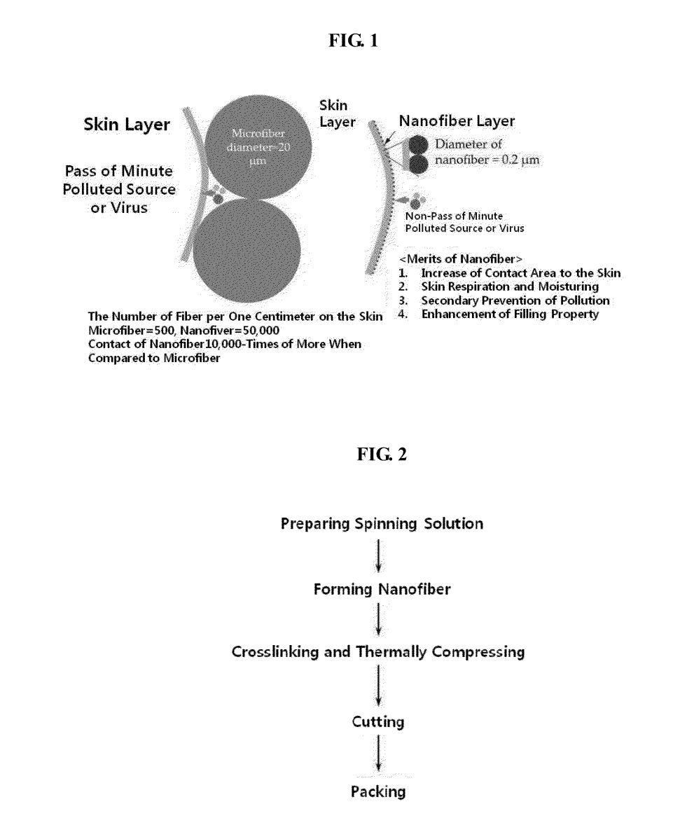

### DESCRIPTION OF THE PREFERRED EMBODIMENTS

A method of manufacturing a cosmetic sheet formed of a nanofiber web according to embodiments of the present invention will be described below with reference to the accompanying drawings.

First, a hydrophilic polymer and a functional material are dissolved in a solvent such as water or alcohol, to thereby prepare a spinning solution. Then, a polymer nanofiber web with a diameter of less than 1 μm is prepared by carrying out electrospinning of the spinning solution.

The hydrophilic polymer nanofiber web undergoes thermal compression or calendaring within a range where alteration or modification of the functional material does not occur, to thereby improve handling property, and is cut and packed in a fit form for an intended use, to thereby obtain a cosmetic sheet formed from nanofiber.

In addition, in some embodiments, a single type water-soluble polymer or a mixture of two or more water-soluble polymers can be blend spun. Here, water and alcohol with compatibility with respect to the polymer may be selected and used as a solvent, alone or a mixture thereof.

In some embodiments, a crosslinking agent is mixed in the spinning solution for crosslinking of the water-soluble polymer. In general, when the polymer material is dissolved, the polymer material is heated and stirred to prepare a spinning solution, the spinning solution is cooled to the room temperature, and thereafter the crosslinking agent is added in the spinning solution. Here, it is preferable that the crosslinking agent should be added in the spinning solution, in a range where a crosslinking reaction does not occur in the spinning solution.

Therefore, in some embodiments, it is preferable that addition of the crosslinking agent at the time of producing the spinning solution should be conducted at the last stage after the water-soluble polymer has been dissolved in water or alcohol, and then the functional material has been added in the spinning solution. The content of the crosslinking agent to be added is preferably in a range of 0.1 wt % to 2 wt %. The crosslinking may preferably employ a method such as hot air, heat-treatment calendering, or UV irradiation. Further, the content of the crosslinking agent and the crosslinking time can be adjusted in accordance with a purpose of an intended use.

A water-soluble polymer material that can be used in some embodiments, may be a synthetic polymer or natural polymer that is a material that can be electrospun. In this case, the synthetic polymer and the natural polymer may be used alone or in combination thereof. However, the polymer material that is dissolved in water or alcohol to thereby form nanofiber by electrospinning may be applied in some embodiments, without any particular limitation.

FIG. 2 is a flowchart for schematically explaining a process of manufacturing a spontaneous melt-type cosmetic sheet formed from nanofiber according to the present invention. The specific process will be described in more detail below with reference to FIG. 2.

Preparation of a Spinning Solution Containing a Hydrophilic Polymer and a Functional Material

A hydrophilic polymer is dissolved in a solvent such as water or alcohol with a spinnable concentration to thus prepare a spinning solution. A concentration capable of maintaining a fibrous form during spinning is suitable as the concentration of the spinning solution, and the polymer material in the spinning solution (that is, the solvent plus the polymer material) is suitable in a range of about 5 to 70 wt %.

When the proportion of the polymer is less than 5 wt %, the drop may occur due to the low concentration rather than forming nanofiber during electrospinning, and as a result no fiber may be often formed. In the case that the proportion of the polymer exceeds 70 wt %, the amount of the polymer is too much to form nanofiber due to defective spinning Therefore, it is necessary to prepare a spinning solution in a suitable concentration range that fiber can be formed in accordance with the type of the polymer applied. In particular, when two or more polymers are blended and spun, the polymer and the solvent should have compatibility, and need to meet the condition that phase separation or the like does not occur. In addition, it is preferred to prepare the spinning solution with a consideration of a difference in the volatilization of the solvent between one kind of a solvent and a mixture of two kinds of solvents.

Further, when a spinning solution is prepared by blend the functional material and the crosslinking agent, it is necessary to prepare the spinning solution by dissolving the polymer material in the solvent and then adding the functional material and the crosslinking agent in the solvent at the room temperature. In the case of using the PVA as the polymer, it is common to manufacture the spinning solution while heating and stirring the spinning solution. Accordingly, since the addition of the crosslinking agent brings about a crosslinking reaction, there are fears that a solution may not be formed but solid matters may be formed.

Forming Nanofiber Webs

The prepared spinning solution is transferred to a spinning nozzle by using a metering pump, and a voltage is applied to the spinning nozzle by using a high-voltage control device, to thereby execute electrospinning. A voltage that can be spun in the range of 2 kV to 100 kV is conducted as the orthovoltage, and a collector plate may be connected to the ground, or may be charged into the negative electrode.

The collector plate is preferably configured to include an electrically conductive metal, or exfoliated paper. It is preferable to use a suction collector attached to the collector plate to smoothen bundling of the fiber during spinning. The distance between the spinning nozzle and the collector plate may be controlled and used in the range of 5 to 50 cm.

The discharge rate of the spinning solution per hole during spinning may be preferably controlled at 0.01 to 5 cc/hole·min using a metering pump, and the spinning solution may be preferably spun in an environment of the relative humidity of 10 to 90% in a chamber where the temperature and humidity can be adjusted during spinning. In particular, the basis weight of the nanofiber may be preferably in the range of 10 to 50 gsm, to improve handleability.

Crosslinking and Thermal Compression (Calendering)

In order to control the time taken to dissolve the electrospun nanofiber web by water or mist, there is a need to perform a crosslinking process, and in order to increase the bonding strength between strands of the nanofiber, it is necessary to perform a thermal compression process.

Complete crosslinking or partial crosslinking proceeds as the crosslinking process, and is adjusted to be spontaneously melted over time by water and mist. Crosslinking can be carried before or after the thermal compression or calendering, or crosslinking may be also carried out simultaneously with calendering. In addition, for the partial crosslinking, the type and content of the crosslinking agent added in the spinning solution, the hot air, the heat treatment calendering, the UV irradiation time, and the like can be adjusted.

Here, the crosslinking is preferably carried out in the range of 80° C. to 100° C. in 30 minutes or less that deterioration or destruction of the functional material does not occur. The crosslinking is conducted. When the calendaring process in 150° C. is executed, the crosslinking is preferably conducted within 30 seconds, in the range that the alteration of the functional material does not occur.

A cosmetic sheet is prepared through the steps of cutting and packing the crosslinked or thermally compressed nanofiber layer according to a fit for the purpose, to thereby complete the present invention.

Hereinafter, the present invention will be described in further detail through examples. However, the following examples are for explaining the present invention in more detail, but do not limit the scope of the present invention to these examples.

### Example 1

A hydrophilic polymer such as polyvinyl alcohol (PVA) was dissolved by 25 wt % in water to have prepared a PVA spinning solution at 80° C. Water-soluble collagen was added by 10 wt % in the prepared PVA solution with respect to the PVA and agitated at the room temperature to thereby have prepared a spinning solution. The spinning solution was moved in a spinning pack, to then have performed electrospinning in the spinning atmosphere of a temperature of 30° C. and a relative humidity of 60%, at an applied voltage of 25 kV, so that a distance between the spinning nozzle and the collector plate was 20 cm, and the discharge rate of the spinning solution per minute became 0.05 cc/hole, to thereby have obtained a nanofiber web.

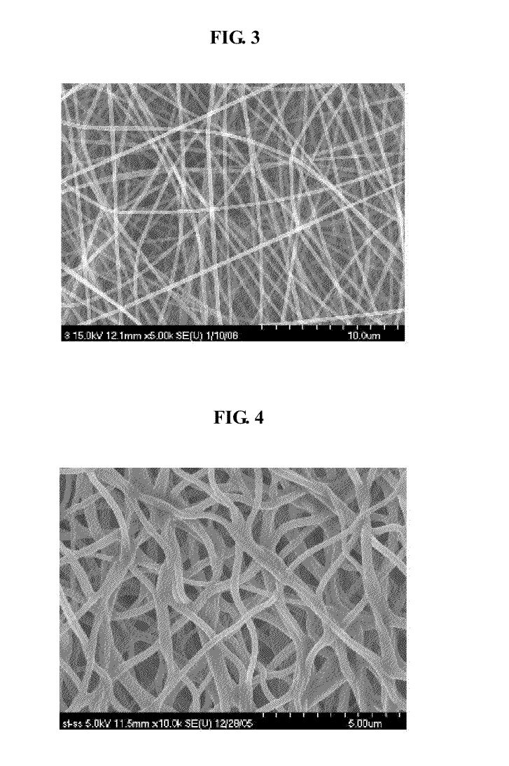

A scanning electron micrograph (SEM) photo of the thus-obtained PVA nanofiber web is shown in FIG. 3, in which distribution of the fiber diameter was approximately 150-350 nm, and the average fiber diameter was about 200 nm. The thus-prepared PVA nanofiber web was treated for 10 minutes by using hot air of 100° C. to thereby have performed partial crosslinking.

### Example 2

A hydrophilic polymer such as polyvinyl alcohol (PVA) was completely dissolved by 25 wt % in water at 80° C., and thereafter a crosslinking agent such as TSA (para-toluene sulfonic acid) and a functional substance such as water-soluble collagen were added in the PVA-dissolved water by 2 wt % and 5 wt %, respectively, with respect to the PVA at the room temperature to thereby prepare a spinning solution. The spinning solution was moved in a spinning pack, to then have performed electrospinning in the spinning atmosphere of a temperature of 30° C. and a relative humidity of 60%, at an applied voltage of 25 kV, so that a distance between the spinning nozzle and the collector plate was 20 cm, and the discharge rate of the spinning solution per minute became 0.05 cc/hole, to thereby have obtained a nanofiber web.

The spinning solution was spun in the same manner as that of Example 1 to thus have obtained a PVA nanofiber web containing the crosslinking agent and the water-soluble collagen.

The thus-prepared PVA nanofiber web was treated by using hot air for 30 minutes at 150° C. to thereby have performed complete crosslinking. It was visually confirmed that the color of the surface of the thus-prepared PVA nanofiber web was deformed from white to yellow in accordance with the crosslinking progress of the surface of the thus-prepared PVA nanofiber web. A scanning electron micrograph (SEM) photo of the PVA nanofiber web having undergone the crosslinking process is shown in FIG. 4. It can be confirmed that fusion has occurred between strands of the fiber by the crosslinking treatment.

### Example 3

A hydrophilic polymer such as polyvinyl pyrrolidone (PVP, K-80) was dissolved by 15 wt % in a mixed solvent of ethanol and water (75/25 wt %), at the room temperature to thereby have prepared a PVP spinning solution. Functional materials such as water-soluble collagen, a hyaluronic acid, and vegetable platinum were added in the PVP solution by 5 wt %, respectively, with respect to the PVP to then be agitated at the room temperature, and a crosslinking agent such as poly (urea-co-formaldehyde) was added by 2 wt % in the PVP solution with respect to the PVP, to thereby have prepared a spinning solution.

The spinning solution was electrospun in the same manner as that of Example 1 to thus have obtained nanofiber web containing the PVP and the functional material. The thus-prepared nanofiber web was crosslinked by using hot air for 30 minutes at 125° C. to thereby have a PVP crosslinked nanofiber web containing the functional material.

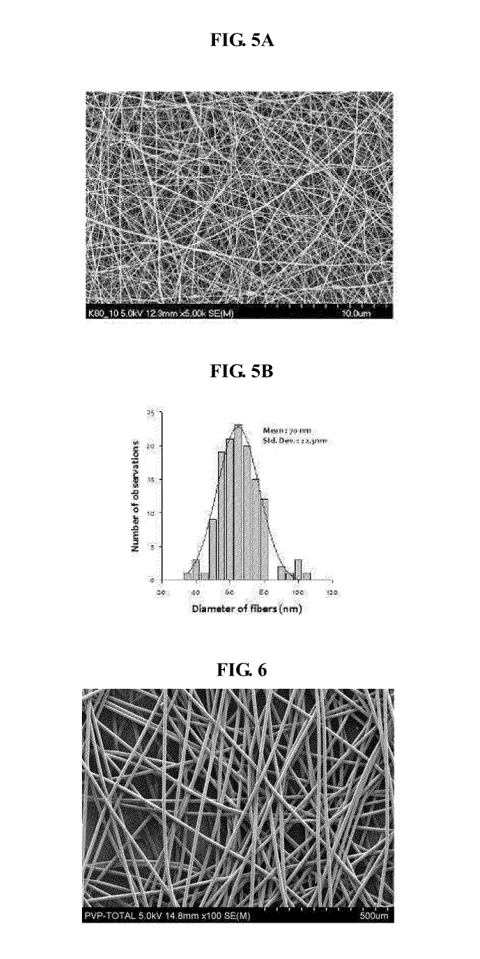

Meanwhile, in the same manner as in Example 1, a PVP-alone nanofiber web on which the functional material was not mounted was prepared. A scanning electron micrograph (SEM) photo and a graph illustrating distribution of a diameter of the nanofiber web are shown in FIGS. 5A and 5B, respectively. An average fiber diameter was about 70 nm.

FIG. 6 shows a scanning electron micrograph (SEM) photo of PVP mounted with a functional material in accordance with the present embodiment, and thus it can be seen that the diameter of the fiber has remarkably increased as compared to the case of the PVP alone.

### Example 4

A hydrophilic polymer such as PVA and PVP was dissolved at a ratio of 50:50 wt % in a mixed solvent of water and ethanol (75/25 wt %), and was stirred at a temperature of 60° C., to thereby have prepare a polymer mixed spinning solution. The prepared polymer mixed spinning solution was cooled to the room temperature and then functional materials such as water-soluble collagen, a hyaluronic acid, vegetable platinum, and tocopherol were mixed by 3 wt %, respectively with respect to the polymer, to thereby have prepared a polymer/functional materials mixed solution.

A crosslinking agent such as TSA was added by 1 wt % in the prepared polymer/functional materials mixed solution with respect to the PVA and was stirred to thereby have prepared a spinning solution. The spinning solution was electrospun in the same manner as that of Example 1. Here, electrospinning was conducted so that the content of PVA/PVP nanofiber was 20 gsm, and calendering was conducted by using a calender roll heated to 150° C. for a contact time of 10 seconds, to thereby have conducted partial crosslinking.

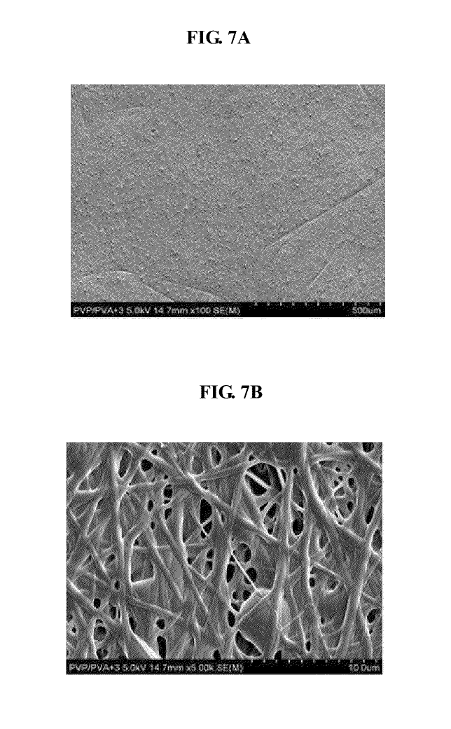

FIGS. 7A and 7B are scanning electron micrograph (SEM) photos respectively having 100-times magnification and 5,000-times magnification of a PVA/PVP composite nanofiber web containing polymer and functional materials produced according to the present example 4. As shown in FIGS. 7A and 7B, it can be confirmed that fusion takes place between strands of the nanofiber by partial crosslinking and calendaring.

### Example 5

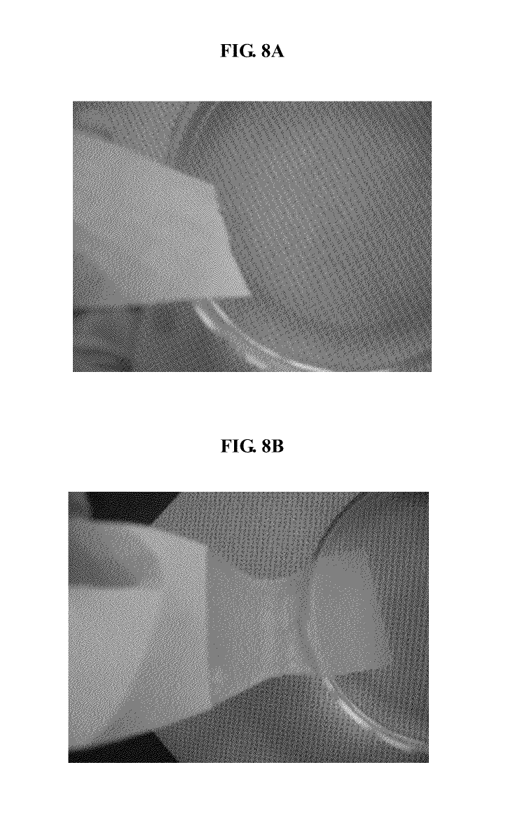

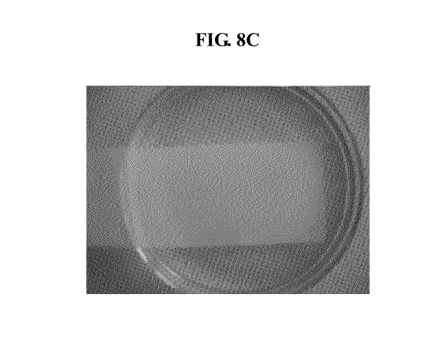

A crosslinking degree of the PVA/PVP nanofiber webs prepared in Example 4 were measured through calendering. Photos of the PVA/PVP nanofiber webs that were obtained by adjusting contact times into 3 seconds, 10 seconds, and 30 seconds, respectively at a calendering temperature of 150° C. have been illustrated in FIGS. 8A, 8B and 8C.

As shown in FIGS. 8A, 8B and 8C, as the contact time became longer during conducting the hot plate calendaring, it can be seen that the crosslinking degree has increased. As shown in FIG. 8A, it can be seen that when the contact time was short, crosslinking did not proceed, and thus the nanofiber was dissolved immediately as soon as the nanofiber contacted water due to the large specific surface area of the nanofiber. As shown in FIGS. 8B and 8C, it can be seen that when crosslinking has proceeded, the nanofiber was not dissolved immediately but absorbed moisture although the nanofiber contacted water.

From these results, in the case of using the cosmetic sheet including the nanofiber web according to the present invention, it can be seen that an effect of properly delivering the efficacious ingredients for a desired time via a crosslinking process is obtained by attaching the cosmetic sheet to the skin after having created a moisture environment in the skin or having sprayed mist on the skin.

As described above, the present invention has been described with respect to particularly preferred embodiments. However, the present invention is not limited to the above embodiments, and it is possible for one of ordinary skill in the art to make various modifications and variations, without departing off the spirit of the present invention. Thus, the protective scope of the present invention is not defined within the detailed description thereof but is defined by the claims to be described later and the technical spirit of the present invention.

### INDUSTRIAL APPLICABILITY

The present invention may be applied to a spontaneous melt-type cosmetic sheet that is prepared by mounting a functional material on a large specific surface area nanofiber, as well as various kinds of medical or green materials.

What is claimed is:

1. A cosmetic sheet with a controlled dissolution velocity obtained by electrospinning a spinning solution that is formed by dissolving a water-soluble polymer material together with a functional material in a solvent, wherein the cosmetic sheet is formed from a nanofiber web having a diameter less than 1 μm and having fine pores.

2. The cosmetic sheet according to claim 1, wherein the water-soluble polymer material contains one or a mixture of two or more selected from a group consisting of PVA (polyvinyl alcohol), PVP (polyvinyl pyrrolidone), PEO (polyethylene oxide), CMC (carboxyl methyl cellulose), starch, PAA (polyacrylic acid) and hyaluronic acid.

3. The cosmetic sheet according to claim 1, wherein the content of the nanofiber is set in a range of 10-50 gsm (gram per square meter).

4. The cosmetic sheet according to claim 1, wherein the functional material employs one or a mixture of two or more selected from a group consisting of a water-soluble collagen, vegetable platinum, tocopherol, xylitol and plant extracts.

5. The cosmetic sheet according to claim 1, wherein the content of the functional material is in a range of 0.5˜50 wt % when compared to the water-soluble polymer.

6. The cosmetic sheet according to claim 1, wherein the solvent employs water or alcohol, or a mixture of water and alcohol.

7. A method of manufacturing a cosmetic sheet with a controlled dissolution velocity, the method comprising the steps of:
preparing a spinning solution by dissolving a water-soluble polymer material and a functional material in a solvent formed of one of or a mixture of water and alcohol;
obtaining a nanofiber web with a diameter of less than 1 μm by electrospinning the spinning solution;
undergoing a crosslinking treatment of the nanofiber web; and
cutting the crosslinked nanofiber web so as to be fitted on shape of the cosmetic sheet.

8. The method of manufacturing a cosmetic sheet of claim 7, wherein the crosslinking treatment comprises calendaring by thermal compression.

9. The method of manufacturing a cosmetic sheet with a controlled dissolution velocity of claim 7, wherein a crosslinking agent is added in the spinning solution.

10. The method of manufacturing a cosmetic sheet with a controlled dissolution velocity of claim 9, wherein the crosslinking agent (or co-crosslinking agent) comprises at least one selected from TSA (para-toluene sulfonic acid), TMPTMA (tri-methylopropane trimethacrylate), DVB (Divinylbenzene), N-(1-Hydroxy-2,2-dimethoxyethyl) acrylamide, N,N′-Methylenebisacrylamide, ethylene glycol diacrylate, Di(ethylene glycol) diacrylate, boric acid, and glutaraldehyde.

11. The method of manufacturing a cosmetic sheet with a controlled dissolution velocity of claim 9, wherein the content of the crosslinking agent is in a range of 0.1 to 2 wt % when compared to the water-soluble polymer.
